(19) United States
(12) Patent Application Publication
(10) Pub. No.: US 2011/0091908 A1
(43) Pub. Date: Apr. 21, 2011

(54) EARLY PROSTATE CANCER ANTIGEN-2: A NOVEL SERUM SPECIFIC MARKER FOR PROSTATE CANCER DETECTION

(75) Inventor: Robert H. Getzenberg, Lutherville, MD (US)

(73) Assignee: University of Pittsburgh - Of the Commonwealth System of Higher Education

(21) Appl. No.: 12/988,502

(22) PCT Filed: Apr. 24, 2009
(86) PCT No.: PCT/US09/02544

§ 371 Date: Jan. 1, 2011

Related U.S. Application Data
(60) Provisional application No. 61/054,005, filed on May 16, 2008.
(60) Provisional application No. 61/048,447, filed on Apr. 28, 2008.

(51) Int. Cl. G01N 33/574

(52) U.S. Cl. 435/7.23; 436/501

(57) ABSTRACT

Early Prostate Cancer Antigen-2 (EPCA-2) is a prostate cancer biomarker, newly identified via proteomics-based examination of alterations in nuclear structure. EPCA-2 shows a higher overall specificity than PSA. Thus, an immunoassay based on antibodies against an epitope of EPCA-2 more accurately differentiates between confined and non-confined disease than PSA or Gleason score.

### STATEMENT REGARDING FEDERALLY SPONSORED RESEARCH

This invention was made with government support under grant CA65463, awarded by the National Institutes of Health. The government has certain rights to this invention.

### BACKGROUND OF THE INVENTION

The discovery of prostate-specific antigen (PSA) more than 25 years ago represented a major advance in the early detection of prostate cancer. Widespread use of PSA screening has resulted in stage migration, early intervention with effective treatments, and decreased morbidity. PSA has limitations, however, a major one being that the test is not specific for prostate cancer. Elevated levels of PSA have been associated with other prostate conditions, such as benign prostatic hyperplasia (BPH) and prostatitis.

As many as 1.8 million men in the U.S. each year undergo prostate biopsies because their PSA test results are considered to be elevated, yet as few as just one in seven of those men will be diagnosed with biopsy proven cancer.

Since its introduction in the 1980s, PSA has been used for prostate cancer detection. In 1990, a cut-off point of 4.0 ng per ml was established for PSA. See Cooner et al., J. Urol. 143: 1146-52 (1990). The use of this cut-point has been questioned, however. Studies have suggested that a PSA level of 2.5 ng per milliliters should be the upper threshold for the normal range. See Thompson et al., JAMA 294: 66-70 (2005); and Catalona, JAMA 277: 1452-55 (1997).

In any event, PSA has changed the face of prostate cancer detection. Today, at the time of presentation few men have metastatic disease. This “stage migration” has been a major advance. The principal limitation of PSA is its lack of specificity for prostate cancer. This has lead to a large number of unnecessary biopsies and confusion as to whom should be treated.

Within the past decade, advances in proteomics have stimulated a search for new biomarkers with increased specificity. Changes in the cell nucleus are hallmarks of cancer. Accordingly, the present inventors have used focused proteomics to profile the nuclear structural elements of prostate cancer cells and, in so doing, have identified new biomarkers for the disease. Differences in the protein components of the nuclear structure have been demonstrated in cancer and normal rat prostate, Getzenberg et al., Cancer Res. 51: 6514-20 (1991), and in a transgenic mouse model for prostate cancer, Leman et al., J. Cell Biochem. 86: 203-12 (2002), as well as in benign prostatic hyperplasia (BPH) and prostate cancer. See Lakshmanan et al., J. Urol. 159: 1354-58 (1998), and Pienta et al., Prostate 23: 61-7 (1993).

In particular, the present inventors previously identified Early Prostate Cancer Antigen (EPCA), which is associated with the nuclear structure, as a highly specific plasma-based marker for prostate cancer. See Paul et al., Cancer Res. 65: 4097-100 (2005). It is expressed throughout the prostate and, hence, represents a “field effect” associated with prostate cancer. The expression of EPCA in the “negative biopsies” of men can help reveal whether prostate cancer is localized or non-confined disease. See Uetsuki et al., J. Urol. 174: 514-18 (2005), and Dhir et al., J. Urol. 171: 1419-23 (2004).

### SUMMARY OF THE INVENTION

Early Prostate Cancer Antigen-2 (EPCA-2) is a new prostate cancer biomarker, identified via proteomics-based examination of alterations in nuclear structure. EPCA-2 is unrelated to EPCA and, since it appears only in the prostate cancer tissue, is not associated with a “field effect.”

The present inventors evaluated EPCA-2 levels in patient populations by detecting EPCA-2 in patient serum samples, using antibodies to two EPCA-2 epitopes, EPCA-2.22 and EPCA-2.19.

Studies with EPCA-2.22 antibodies were performed with a study population of 178 men: those with PSAs<2.5 ng per milliliter; those with PSAs>2.5 ng per milliliter who had repeated negative biopsies; men with benign prostatic hyperplasia; men with organ-confined prostate cancer and those with extracapsular disease. Evaluation was effected by means of an enzyme-linked immunosorbent assay (ELISA), using an anti-epitope antibody, EPCA-2.22. The inventors also examined serum samples from a diverse group of controls (n=134). The results were compared with the PSA values from these. Individuals, and a pilot set of samples established cut-off values. From these results, EPCA-2 was found to compare favorably to prostate specific antigen (PSA), in sensitivity and specificity, for detecting prostate cancer. Moreover, EPCA-2.22 was found to differentiate aggressive versus non-aggressive cancers.

Similar studies were performed with respect to EPCA-2.19, and these studies yielded nearly identical results to the EPCA-2.22 studies. Namely, that EPCA-2.22 was found to be a highly sensitive (91%) and specific (100%) marker for detecting prostrate cancer and differentiating prostate cancer from benign prostatic hyperplasia (BPH), other benign conditions and cancer types, as well as normal individuals.

In sum, these results indicate that EPCA-2, as detected by antibodies to two EPCA-2 epitopes, is a highly specific and sensitive marker for detecting prostate cancer.

### BRIEF DESCRIPTION OF THE DRAWINGS

FIG. 1. EPCA-2.22 Levels in Serum Samples Collected from 178 Men and 134 Control Individuals. A total of 312 serum samples were screened for EPCA-2.22. Serum samples were collected from normal men with PSA<2.5 ng per milliliters, normal men with negative biopsies of prostate cancer but PSA>2.5 ng per milliliters, men with highly symptomatic BPH (AUA score>20; mean AUA score=24), men with either non-organ-confined prostate cancer or organ-confined prostate cancer, as well as control populations with various benign and cancerous diseases. Samples were plated in duplicates and in random orders. Utilizing a pilot set, indirect ELISA assays show that EPCA-2.22 has a cut-off point of 30 ng per milliliters and above at the estimated concentration levels.

FIG. 2. EPCA-2.19 Levels in Serum Samples Collected from 194 Men and 134 Control Individuals. A total of 328 serum samples were screened for EPCA-2.19. Serum samples were collected from normal men with PSA<2.5 ng per milliliters (n=43), normal men with negative biopsies of prostate cancer but PSA>2.5 ng per milliliters (n=30), men with highly symptomatic BPH (AUA score>20) (n=33), men with non-organ-confined prostate cancer (n=45), and men with organ-confined prostate cancer (n=43), as well as control populations with various benign and cancerous diseases (n=134). Samples were plated in duplicates and in random orders. Utilizing a pilot set, indirect ELISA assays show that EPCA-2.19 has a cut-off point of 0.5 ng per milliliters and above at the estimated concentration levels.

FIG. 3. Receiver Operator Characteristic (ROC) Curves for EPCA-2.22. (A) EPCA-2.22 has a specificity of 100% (area under the curve 1.00, 95% confidence interval 0.98 to 1.00) in separating men without prostate cancer (PSA levels < and >2.5 ng per milliliters) from those with both organ-confined and non-organ-confined prostate cancer. Total PSA has a specificity of 54% (area under the curve 0.67, 95% confidence interval 0.58 to 0.77) in separating the men without any evidence of prostate cancer (normals) (PSA<and >2.5 ng per milliliters) from those with the combined prostate cancer group. (B) ROC curves also demonstrate that EPCA-2.22 has a specificity of 100% (area under the curve 1.00, 95% confidence interval 0.98 to 1.00) in separating normal men with PSA>2.5 ng per milliliters and negative biopsy of prostate cancer from men with both organ-confined and non-organ-confined prostate cancer, whereas total PSA has a specificity 0% (area under the curve 0.37, 95% confidence interval 0.26 to 0.49) in separating normal men from overall prostate cancer population. (C) When comparing men without prostate cancer and men with BPH from those with prostate cancer, EPCA-222 has a specificity of 92% (area under the curve 0.98; 95% confidence interval 0.96 to 0.99), whereas PSA has a specificity of 65% (area under the curve 0.77; 95% confidence interval 0.69 to 0.84).

FIG. 4. EPCA-2.22 versus PSA in Separating Organ-Confined from Non-Organ-confined Prostate Cancer. In addition to its high sensitivity and specificity, EPCA-2.22 also is able to differentiate men with organ-confined from those with non-organ-confined prostate cancer (area under the curve 0.89; 95% confidence interval 0.82 to 0.97), whereas PSA is not (area under the curve 0.63; 95% confidence interval 0.50 to 0.75).

FIG. 5. Receiver Operator Characteristic (ROC) Curves for EPCA-2.19. EPCA-2.19 has a specificity of 100% and a sensitivity of 91% in separating normal men with PSA<and >2.5 ng/ml from those with prostate cancer. Receiver Operator Curve (ROC) analyses of the EPCA-2.19 assay show an area under the curve (AUC) of 0.982 (95% CI 0.952-0.996, p<0.0001).

FIG. 6. Serum EPCA-2 Levels Before and After Radiation Therapy (RT) and/or Short-Term Androgen Deprivation (AD). EPCA-2 levels decreased significantly in patients undergoing RT and AD or RT alone. EPCA-2 levels did not change significantly in patients undergoing short-term AD alone.

FIG. 7. Longitudinal Analysis of Serum EPCA-2 Levels After Radiation Therapy (RT). EPCA-2 levels decreased following RT therapy.

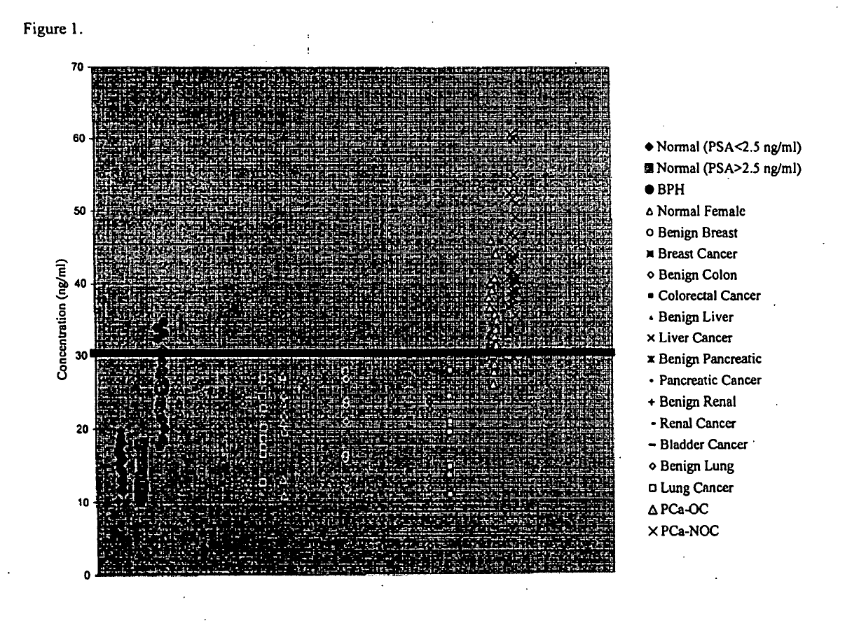

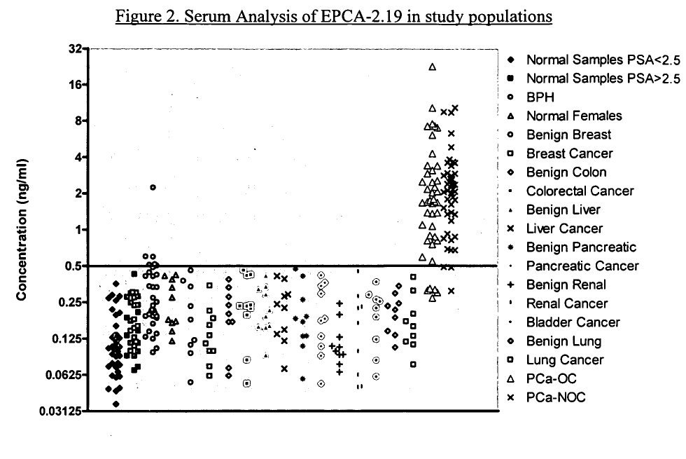

### DETAILED DESCRIPTION OF THE PREFERRED EMBODIMENTS

### Isolation of Nuclear Matrix Proteins

### Fractionation

Nuclear matrix proteins (NMPs) were extracted from prostate cancer tissue, adjacent tissue from these individuals, and donor patients according to the method of Getzenberg et al. (1991), supra. In summary, tissues were finely minced into small pieces and homogenized with a Teflon pestle on ice with 0.5% Triton X-100 in a solution containing 2 mM vanadyl ribonucleoside (Rnase inhibitor) to release the lipids and soluble proteins. The homogenized tissue then was filtered through a 350 μm nylon mesh. DNAse and RNAse treatments were used to remove the soluble chromatin. The remaining fraction contained intermediate filaments and NMPs. This fraction then was disassembled with 8 M urea, and the insoluble components consisting of carbohydrates and extracellular matrix were pelleted. After dialyzing the urea out, the intermediate filaments were allowed to reassemble and were subsequently removed by centrifugation. The NMPs then were precipitated in ethanol. The protein concentration was determined by resuspending the pellet in 2D sample buffer consisting 9 M urea, 65 nM.3-((3-cholamidopropyl)-dimethyl-ammonio)-1propane-sulfonate, 2.2% amhpolytes and 140 mM DTT, and quantitated by Coomassie Plus protein assay (Pierce Chemical Co., Rockford, Ill.) with bovine serum albumin as a standard. The final pellet containing these proteins represent<1% of the total cellular proteins.

### Two-Dimensional Electrophoresis

High resolution, two-dimensional electrophoresis was performed using the Investigator 2-D gel system, product of Genomic Solution (Ann Arbor, Mich.), as described by Getzenberg et al. (1991), supra, and Patton et al., Biotechniques 8: 518-27 (1990). One hundred micrograms of protein were loaded per gel onto a capillary-size IEF column. One-dimensional isoelectric focusing was carried out for 18,000 volt-hours, using 1 mm×18 inch tube gels after 1.5 hours of prefocusing. The tube gels were extruded and placed on top of 1 mm SDS Duracryl (Genomic Solution), high tensile-strength PAGE slab gels. The gels were electrophoresed at 12° C. constant temperature for 4.5 to 5 hours. Gels were fixed with 50% methanol and 10% acetic acid. After thorough rinsing and rehydration, gels were treated with 5% glutaraldehyde and 5 mM DTT after buffering with 50 mM Phosphate (pH 7.2). The gels were stained with silver stain via the methodology of Wray et al., Analytical Biochem. 118: 197-203 (1981).

Molecular weights of the prostate NMPs were identified using standards provided by Genomic Solutions. Isoelectric points (PI's) were determined by means of carbamylated standards (BDH), distributed by Gallard-Schlessinger (Carle Place, N.Y.) and Sigma Chemical Co. (St. Louis, Mo.). EPCA-2 was found to have a MW of 40 kD and a pI of 5.91. Multiple gels were run for each sample, and multiple samples were run at different times. Only protein spots clearly and reproducibly identical in all the gels of a sample type were taken into account as those representing the described NMP's. The gels were analyzed using the BioImage 2D Electrophoresis Analysis System, product of BioImage (Ann Arbor, Mich.), which matches protein spots between gels and sorts the gels and protein spots into a database.

### Protein Sequencing

Sequencing was effected of proteins isolated from spots in two-dimensional gels, as described above. In order to purify and concentrate sufficient quantities of EPCA-2 for sequencing, protein was isolated according to an adaptation of a technique reported by Gavaert et al., “New strategies in high sensitivity characterization of proteins separated from 1-D or 2-D gels,” in: METHODS IN PROTEIN STRUCTURE ANALYSIS 15-26, Atassi, M. Z., and E. Appella (eds.), Plenum Press (New York, 1995). The two-dimensional gels were negatively stained by incubating them in 0.2 M imidazole for 15 minutes, washed several times with deionized water, and stained with warm 0.3 M zinc chloride. Deionized water was used to stop the staining, and the protein gel spots were excised and frozen at −80° C. The spots then were thawed, pooled, and mixed with 0.25% Coomassie blue stain (45% methanol/9% acetic acid) for 20 minutes. With constant agitation, the spots were destained with destaining solution (5% methanol/7.5% acetic acid) for 1 hour, washed with deionized water for 1 hour, and equilibrated in sample buffer (1% SDS/10% glycerol/50 mM DTT/12 mM Tris-HCl pH 7.1) for 1 hour before loading into the acrylamide/agarose gel.

The spots then were concentrated on a mini-agarose/acrylamide gel. The construction of the mini-agarose gel consisted of two pre-warmed (60° C.) glass plates (10 cm×9 cm), separated by spacers 1 cm wide and 1.5 mm thick. A strip of Whatman 3 MM paper was inserted at the bottom to serve as a support for the lower agarose gel, preventing the gel from slippage during electrophoresis. The sample well was formed by a 2 cm wide×1.5 cm thick spacer set between two parallel spacers, each 1 cm wide×1.5 cm thick inserted at the center of the glass plates and attached with adhesive tape at the top edge of the back plate.

The lower gel consisted of a 2 cm-deep agarose gel (1.45% agarose in 0.36 M Tris-HCl pH 8.7/0.1% SDS). Once the agarose had set, it was overlayed with the polyacrylamide stacking gel (5.45% acrylamide/0.13% bisacrylamide/0.12 M Tris-HCl pH 6.8/0.1% SDS). When the stacking gel had set, the central spacer was removed, leaving a well 2 cm high, 2 cm wide, and 1.5 mM thick. The mini concentration gel then was mounted on a small electrophoresis tank, product of BioRad (Hercules, Calif.), and the slot was filled with the equilibrated 2-D gel spots. The remaining volume was filled with blank gel pieces.

The gels were run at 100 V, allowing the proteins to elute out of the combined gel pieces and into the acrylamide. At this time, the central spacer was re-inserted into the sample well until the dye front passed the two parallel 1 cm wide spacers. At that point, the central spacer was removed and electrophoresis continued until the dye front entered the agarose and reached the filter paper.

The agarose section of the gel was fixed in fresh 50% methanol/10% acetic acid shaking, at room temperature for 30 minutes. The gel was stained with 0.05% Coomassie blue stain (50% methanol/10% acetic acid) for 5 minutes and then was destained in 5% methanol/7% acetic acid for 2 hours with constant agitation. The protein band visualized in this manner was excised in a minimal volume of agarose gel, transferred into an sterile tube, and sent for peptide sequencing (Department of Biochemistry, Michigan State University).

Standard approaches were utilized for protein sequencing. Thus, the isolated proteins were subjected to trypsin digestion, and the resulting fragments were separated by HPLC. These HPLC peaks then were subjected to Edman degradation sequencing. The amino acids with the highest degree of confidence were recorded. For some of the amino acids, some degree of ambiguity resulted, which is typical for this process. Searches were performed of the protein databases for all combinations of potential amino acid combinations. Sequences were obtained from three of the resulting HPLC peaks (Peak 4, Peak 19, and Peak 22). These sequences were utilized to produce antibodies against them, as described in the next section.

### Antibody Production

A standard protocol was followed in the production of antibodies raised against the EPCA-2 peptides. Utilizing the peptide sequence derived from the corresponding spots from high resolution two-dimensional gels, peptides were designed from which to raise antibodies. These peptide sequences were chosen based upon the length of the sequence obtained. The peptides produced were modified slightly to include the addition of terminal cysteines for coupling purposes. The sequences were verified through mass spectroscopy and conjugated to keyhole limpet hemocyanin (KLH). The resulting antigens were suspended in saline and emulsified by mixing with an equal volume of Freund's Adjuvant. Two New Zealand white rabbits (3-9 months old) were injected with the peptide into three to four subcutaneous dorsal sites four times over a three month period. The animals were bled from the auricular artery and the serum collected from three production bleed. Antibodies were produced by Cocalico Biologicals Inc. (Reamstown, Pa.) under an Institutional Animal Care and Use Committee approved protocol. Two animals were utilized for the production of antibodies against each of the peptides.

### Evaluation of Anti-Peptide Antibodies

Peptide sequence data, obtained as described above, were employed to raise polyclonal antibodies against KLH-linked synthetic peptides representing three putative epitopes: VIQPYPNFYMV (EPCA-2.22); FAQDNDL (EPCA-2.19); and SFGQVK (EPCA-2.4). Pursuant to the methods set out in U.S. Pat. No. 6,617,432, the contents of which are incorporated by reference here, antibodies were produced against each of three peptides corresponding, respectively, to these epitopes.

Immunoblot and ELISA analysis detected the EPCA-2 protein in human prostate cancer but not in normal adjacent tissue or in normal tissue from organ donor prostates (data not shown). Two-dimensional immunoblots confirmed that the antibodies did detect the original spot.

This analysis was done on a 10% gel versus the 12% of the original gel. On the 10% gel, the resolution of the molecular weights in this range is lower than on the 12% gel. In this analysis, it appeared that the proteins were smaller than the observed 40 kDa. The increased sieving properties of a 12% gel make them more appropriate for separation of smaller proteins in the range of 40 kDa and less. This makes molecular weight determinations in that range more accurate than with the 10% gels.

In order to clarify this point, both types of samples were run on a single 12% gel. For both rat and humans, EPCA-2 was revealed to be approximately 40 kDa (data not shown). The rat protein was a slightly higher molecular weight than the human versions of the protein, indicating that the two proteins are similar in size but not identical.

Screening of antibodies against all three EPCA epitopes consisted of a pilot set of 30 serum samples: 10 normals, 10 organ-confined prostate cancers, and 10 non-organ-confined prostate cancers. The pilot data demonstrated that antibodies for both EPCA-2.19 and 2.22 comprised assays with the highest sensitivity and specificity in separating men with prostate cancer from those without. The pilot data also demonstrated that antibodies against both EPCA-2.19 and 2.22 were similar and complementary to one another in terms of their sensitivity and specificity. Accordingly, the specific data related below are from indirect ELISA assays for EPCA-2.22 and EPCA-2.19.

In addition, the pilot data were used to establish the optimal conditions and cut-off points for the ELISA assays for EPCA-2.22 (30 ng per milliliters) and EPCA-2.19 (0.5 ng per milliliters). Since the recombinant protein for the EPCA-2 was unavailable, concentrations that correspond to these absorbance levels were estimated by the equation that resulted from fitting various concentrations of the EPCA-2.22 and EPCA-2.19 peptides to a sigmoidal curve using Graph Pad Prism 4 software. The results presented below do not include the values obtained from the samples that were used as the pilot set. The inventors measured inter-assay variability, running repeated samples on the assay results in values that are within ten percent of one another.

After establishing the cut-offs using the pilot sets, the EPCA-2 ELISA assay was used to measure the level of EPCA-2.22 and EPCA-2.19 in the sera of the study populations. Table 1A depicts the characteristics for the group of 178 male subjects for the EPCA-2.22 assay, and Table 1B depicts the characteristics for the group of 134 male subjects for the EPCA-2.19 assay. In addition to evaluating these men, additional control populations were provided to us for analyses. For the EPCA-2.22 assay, the control population consisted of 134 serum samples from female patients, as well those with a variety of benign disease and cancers. These samples were selected to represent a diverse series of control samples (Table 1C). For the EPCA-2.19 assay, the control population also consisted of 134 serum samples, but from both male and female patients. The patients were healthy females and males and females with a variety of benign disease and cancers. These samples were selected to represent a diverse series of control samples (Table 1D).

Analysis of the 178 men showed that the EPCA-2 assay was highly specific in discriminating between individuals with and without prostate cancer. The results for the EPCA-2.22 and EPCA-2.19 assays are discussed below.

Men with no evidence of disease, regardless of their PSA levels, as well as the control group of patients with other cancer types and benign conditions all have their serum samples below the EPCA-2.22 cut-off, whereas 8 of 35 BPH patients have a level of EPCA-2.22 higher than the cut-off point. The overall specificity for EPCA-2.22 was 97%. The mean levels of EPCA-2.22 from all the patients screened in this study were compared with total PSA levels and other characteristics (Tables 1A and 1C).

In accordance with the present invention, an EPCA-2 assay also was highly sensitive in the detection of men with prostate cancer. Using the cut-off, EPCA-2.22 detected 36 of 40 men with organ-confined prostate cancer and 39 of 40 men with non-organ-confined disease, indicating that EPCA-2.22 has a sensitivity of 90% for organ-confined prostate cancer, 98% for non-organ-confined prostate cancer, and 94% for the combined prostate cancer group, respectively (Table 2A).

To determine the effects of removal of the prostate cancer by surgery on serum EPCA-2 levels, samples were obtained from 10 men both at the time of their surgery as well as between 8 to 24 months after. All 10 had EPCA-2.22 levels that were positive prior to surgery. Nine of these 10 individuals had EPCA-2.22 levels that were considered to be normal after their prostatectomies. The mean EPCA-2.22 levels before (31.99±0.89 ng per milliliters) and after (25.17±3.26 ng per milliliters) surgery were statistically different (Table 2B, p=0.0002). Table 1E depicts the serum levels of both EPCA-2.22 and total PSA for this group of men before and after prostatectomy.

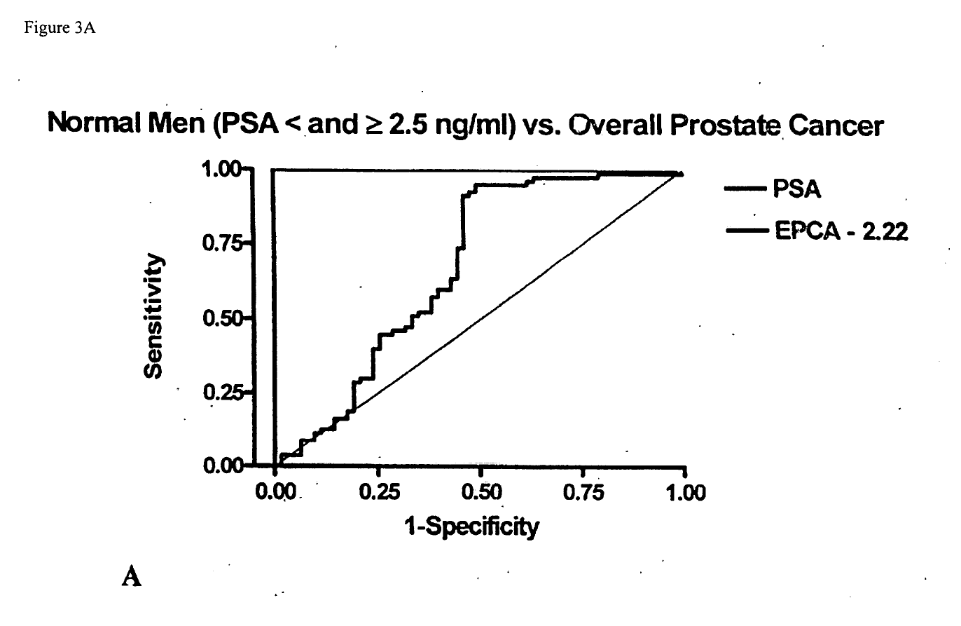

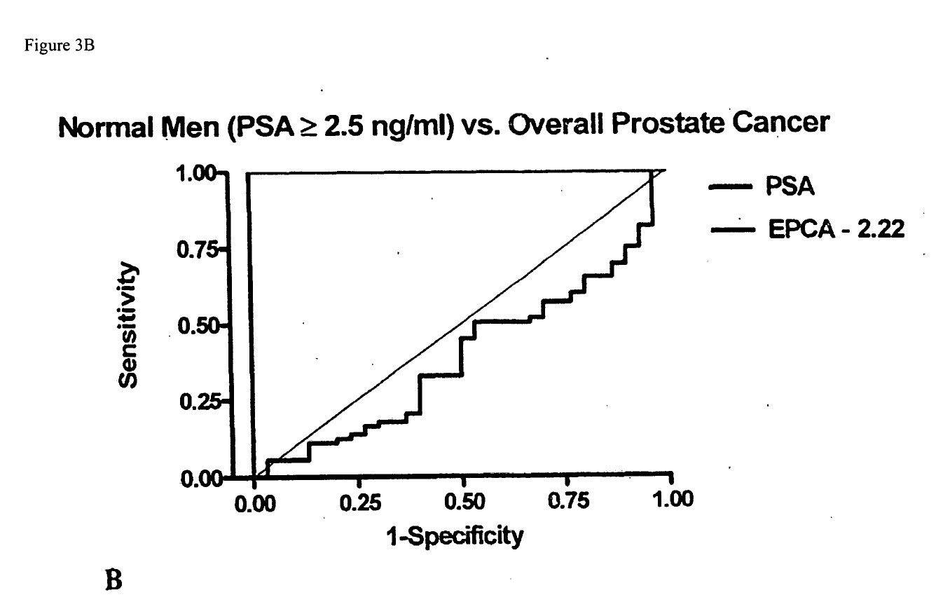

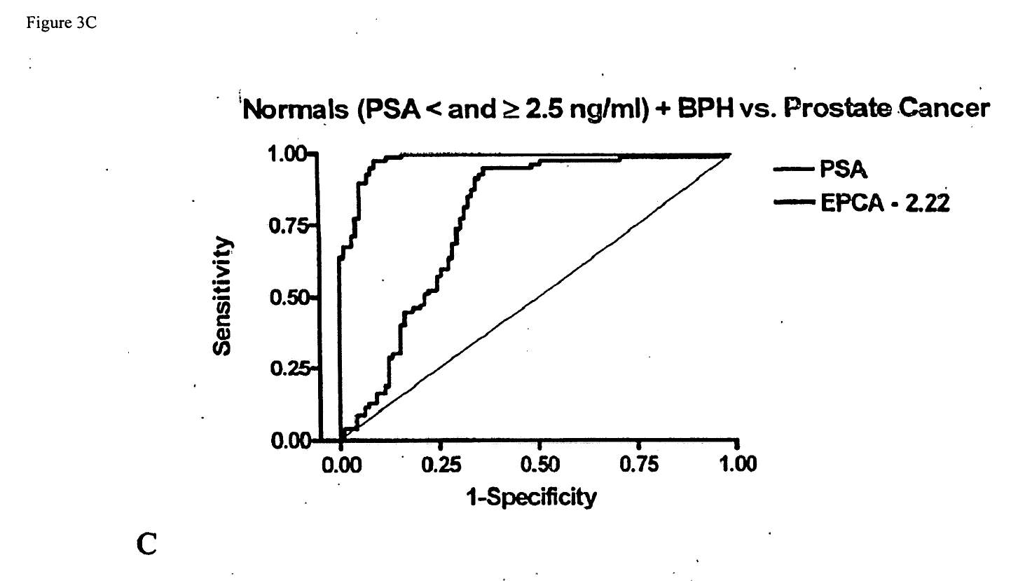

The Receiver Operator Characteristic (ROC) curves were assessed, as shown in FIG. 3A. Total PSA has a specificity of 54% (area under the curve 0.67; 95% confidence interval 0.58 to 0.77) in differentiating patients in the screening population with no evidence of disease from those with prostate cancer. In contrast, EPCA-2.22 has a specificity of 100% (area under the curve 1.00, 95% confidence interval 0.98 to 1.00) in separating men without prostate cancer (PSA levels<and >2.5 ng per milliliters) from the combined group with the disease. The ROC curves also demonstrate that EPCA-2.22 (FIG. 3A) has a specificity of 100% (area under the curve 1.00; 95% confidence interval 0.98 to 1.00) in separating men with PSA>2.5 ng per milliliters who underwent repeated negative biopsies for prostate cancer from those with prostate cancer, whereas total PSA fails to separate these groups (area under the curve 0.37; 95% confidence interval 0.26 to 0.46). Furthermore, the ROC curves (FIG. 3A) also show that EPCA-2.22 has a specificity of 92% (area under the curve 0.98; 95% confidence interval 0.96 to 0.99) in separating men without prostate cancer and men with BPH from those with prostate cancer, whereas PSA has a specificity of 65% (area under the curve 0.77; 95% confidence interval 0.69 to 0.84). The overall specificity and sensitivity for EPCA-2.22 are summarized in Table 2A. In addition, EPCA-2.22 levels using the cut-off of 30 ng per milliliters was statistically more accurate in separating men with prostate cancer from those without (Table 2B, p=0.002) when compared to PSA (at 2.6 ng per milliliters; sensitivity and specificity were 81% and 41%, respectively). See Thompson et al., JAMA 294: 66-70 (2005). These studies demonstrate that the EPCA-2.22 serum-based assay is more specific and sensitive in the detection of prostate cancer than total PSA levels.

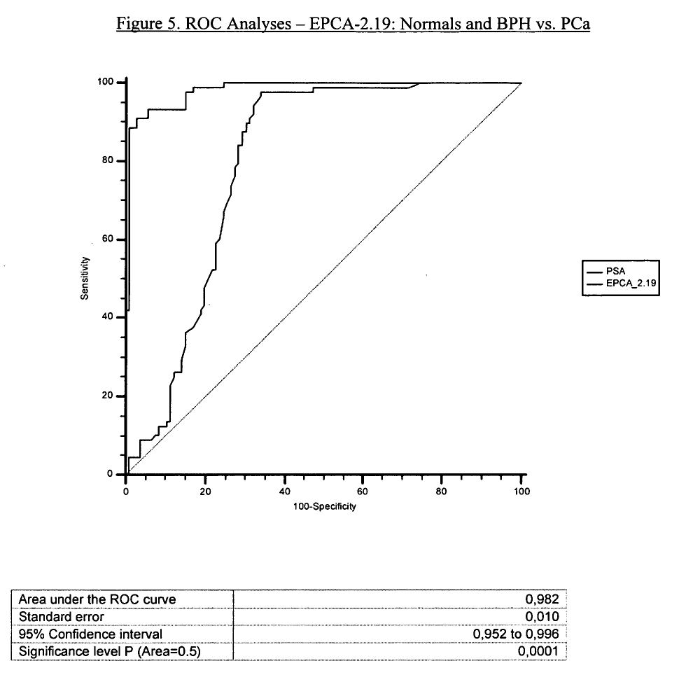

The results of studies on the EPCA-2.19 serum-based assay were very similar to the EPCA-2.22 results. See FIG. 2. Notably, EPCA-2.19 has a specificity of 100% and a sensitivity of 91% in separating normal men with PSA<and >2.5 ng/ml from those with prostate cancer when using a 0.5 ng per milliliter cut-off ROC analyses of the EPCA-2.19 assay show an area under the curve (AUC) of 0.982 (95% CI 0.952-0.996, p<0.0001), as shown in FIG. 5. Table 3 provides further details on the specificity and sensitivity achieved using EPCA-2.19 with different patient populations and with different cut-offs.

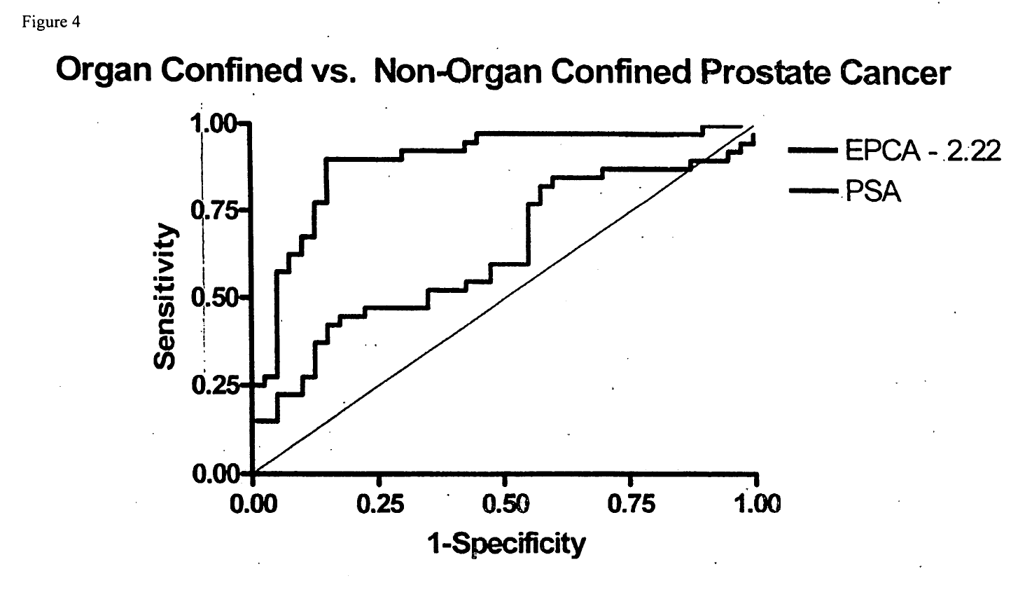

A major issues relating to prostate cancer is the differentiation of aggressive prostate cancer from less aggressive disease. As shown in FIG. 4, the inventive EPCA-2.22 assay was able to separate men with organ-confined prostate cancer from those with non-organ-confined disease (area under the curve 0.89, 95% confidence interval 0.82 to 0.97; p<0.0001). In contrast, total PSA does not separate the two, types of prostate cancer (area under the curve 0.63; 95% confidence interval 0.50 to 0.75; p>0.05) and the Gleason scores between the groups are statistically identical.

Using a cut-off of 30 ng per milliliters, the EPCA-2.22 assay has a 94% overall sensitivity and 100% specificity (area under the curve 1.0; 95% confidence interval 0.98 to 1.00). The specificity for PSA is 54% (area under the curve 0.67; 95% confidence interval 0.58 to 0.77). In addition, EPCA-2.22 differentiates between localized and extracapsular disease (area under the curve 0.89; 95% confidence interval 0.82 to 0.97; p<0.001), whereas PSA does not (area under the curve 0.62; 95% confidence interval 0.50 to 0.75; p=0.05).

Thus, EPCA-2, as measured by either anti-EPCA-2.22 or anti-EPCA-2.19 aitbodies, shows a higher overall specificity than PSA. EPCA-2 more accurately differentiates between confined and non-confined disease than PSA or Gleason score.

The use of novel approaches (i.e., proteomics) to develop more specific biomarkers to detect prostate cancer has permitted us to focus on the molecular correlates of what the pathologist sees. Through this search, we were able to identify EPCA-2 and to demonstrate its utility as a serum-based biomarker for the detection of prostate cancer. The assay of the invention was characterized by a high specificity for a number of benign conditions.

In addition a high overall specificity, a biomarker in this context ideally should also differentiate between aggressive and non-aggressive prostate cancer. As detailed below in a section on methodology, analysis of the pilot set of 30 samples on both of the EPCA-2 epitopes, EPCA-2.19 and 2.22, showed almost identical and complementary results in sensitivity and specificity. Yet, the mean levels of EPCA-2.22 were significantly higher in men with non-confined prostate cancer (42.81±6.74 ng per milliliters) than those with organ-confined disease (33.90±4.18 ng per milliliters) (p<0.0001, Table 2B). This difference persists even though the mean Gleason scores and total PSA levels do not vary between men with organ-confined versus extracapsular disease. The individuals in this study are representative of the types of prostate cancer patients presenting today.

The present invention provides what, to the inventors' knowledge, is the first serum-based marker assay to differentiate aggressive versus non-aggressive cancers. Discovering that two epitopes of the same protein result in similar types of assays provides further support for the validity of this biomarker. These small differences may reflect the three-dimensional folding on the protein and its reactivity within serum.

The EPCA-2 serum assay of the invention not only was able to distinguish between normal men and all cases of prostate cancer combined but also was able to differentiate patients with other types of cancers and benign conditions. EPCA-2.22 also had a sensitivity that was significantly higher than that of PSA. Taken together, the findings detailed above demonstrate that EPCA-2, a prostate cancer-associated nuclear protein, can be utilized as a serum-based biomarker for prostate cancer.

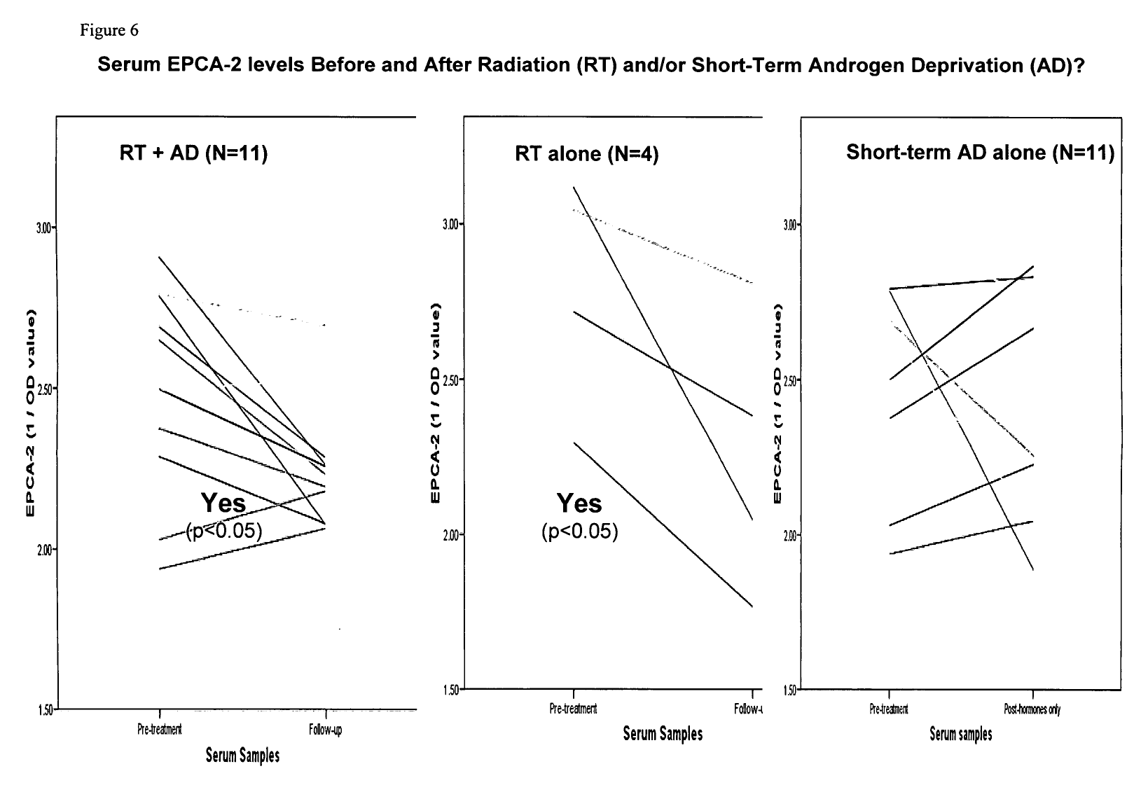

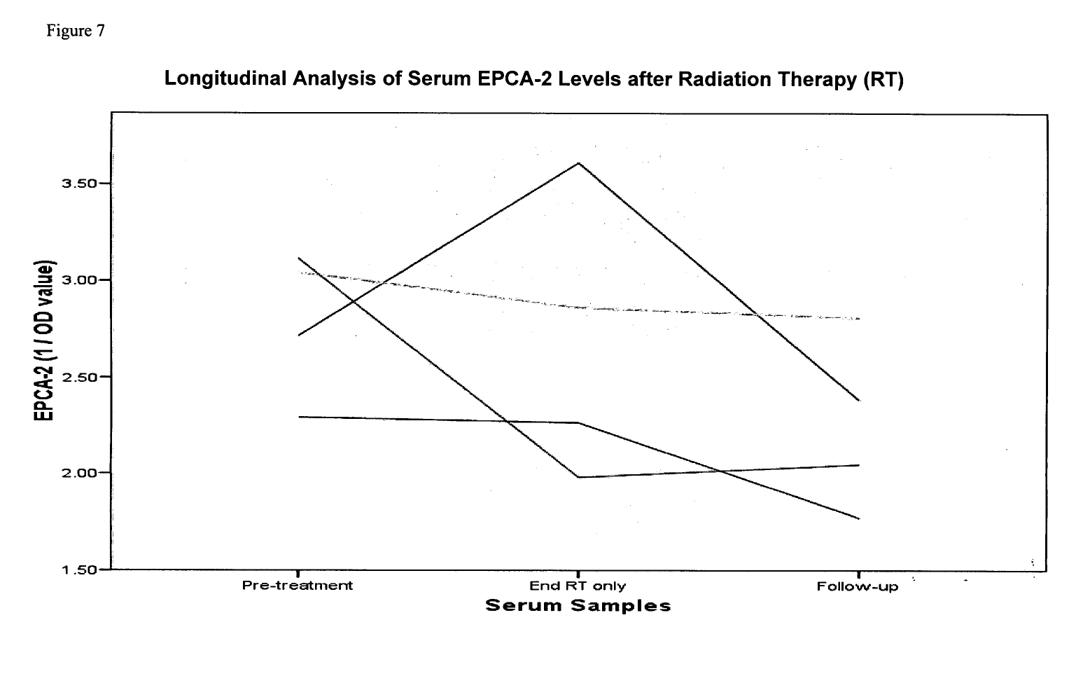

A biomarker that could track the response to prostate cancer to treatment would also be beneficial. To test whether levels EPCA-2 could correlate with disease course in men with prostate cancer, serum EPCA-2 levels were measured in men undergoing short term androgen deprivation (AD) therapy and then subsequent radiation therapy (RT). Specifically, pre-treatment and follow-up EPCA-2 levels were measured in men undergoing both AD and RT (n=11), RT alone (n=4), and short-term AD alone (n=11). FIG. 6 shows the results of the tests. EPCA-2 levels decreased significantly in patients undergoing RT and AD or RT alone. EPCA-2 levels did not change significantly in patients undergoing short-term AD alone. Another test, the results of which are shown in FIG. 7, shows the EPCA-2 levels in four patients pre-treatment, at the end of RT, and at follow-up times. Consistent with FIG. 5, there was a decrease in EPCA-2 levels following RT. These results indicate that EPCA-2 could be used to track disease course. Further details appear below on the methodology employed by the inventors in the work described above. These details and the related methodology are illustrative only and not limiting of the present invention.

### Study Populations

### A. EPCA-2.22

Serum samples were collected from five different groups of men (n=178) under an IRB approved protocol from consented patients (supported by the NCI Early Detection Research Network). The first group consists of 33 normal men with PSA<2.5 ng per milliliters. This group of men served as part of a prostate cancer screening program performed by the Brady Urological Institute of the Johns Hopkins Hospital. These individuals were not previously detected as having abnormal PSA levels and have no clinical evidence of prostate cancer. Sera were also collected from 30 men with PSA levels greater than or equal to 2.5 ng per milliliters who underwent repeated biopsies at the Brady Urological Institute all of which were negative for prostate cancer. The third group consisted of 35 men with normal PSA levels that were biopsied with highly symptomatic BPH (AUA symptom score>20, mean score=24). The last two groups were from 40 men with organ-confined prostate cancer and 40 men with non-organ-confined prostate cancer. These are men who had positive biopsies for prostate cancer and underwent radical prostatectomy at Johns Hopkins. Sera from these groups of men were collected just prior to surgery. Men with organ-confined prostate cancer are classified as those without any extracapsular extension, seminal vesicle invasion or lymph node metastases. Men with non-organ-confined prostate cancer are those with at least one of the above-described pathologic descriptors. The population surveyed consisted principally of Caucasians (90%). Profiles of these patients are outlined in Table 1A.

In addition to the groups mentioned above, the inventors also screened serum samples from a group of 10 men who underwent radical prostatectomy for prostate cancer who had serum samples obtained before the surgery and approximately 8 to 24 months after surgery. Profiles of these men and their serum levels are listed in Table 1E.

To validate our findings, an additional sample set was analyzed. This group consisted of 134 serum samples from normal females, individuals with benign breast disease and breast cancer, benign lesions of the colon and colorectal cancer, benign liver disease and liver cancer, benign pancreatic disease and pancreatic cancer, benign renal disease and renal cancer, bladder cancer, as well as benign lung disease and lung cancer patients. These patients were diagnosed with non-metastatic cancer and their serum samples were obtained just prior to surgery when applicable. Profiles of the patients from the control group are listed in Table 1B.

### B. EPCA-2.19

Serum samples were collected from consented patients (n=134) under an IRB approved protocol (supported by the NCI Early Detection Research Network). Five different groups of serum samples were obtained for this study. The first group consists of serum samples from 43 normal men with no clinical evidence of prostate cancer and PSA levels<2.5 ng/ml. This group of men is part of a prostate cancer screening program. The second group consists of 30 normal men with PSA>2.5 ng/ml but have repeated negative biopsy for prostate cancer. The last three groups consist of 43 men with organ confined prostate cancer, 45 men with non-organ confined prostate cancer, and 33 men who were biopsied with highly symptomatic BPH (AUA symptom score>20). Men with non-organ confined prostate cancer are classified as those without any extracapsular extension, perineural invasion or seminal vesicle invasion, whereas men with non-organ confined prostate cancer are those with either extracapsule extension, invasion of the lymph nodes or seminal vesicle. A total of 194 serum samples were screened for EPCA-2. Profiles of the patients screened for EPCA-2.19 are outlined in Table 1B.

Additional control groups consisted of serum samples from normal females, women with benign breast disease and breast cancer, benign and colorectal cancer, benign and liver cancer, benign and pancreatic cancer, benign and renal cancer, bladder cancer, as well as benign and lung cancer patients. The cancer patients were diagnosed with non-metastatic disease and their sera were collected prior to surgery. Profiles of the patients from the control group are listed in Table 1D. The serum samples were then stored at −80° C. before the EPCA-2.19 ELISA assays were performed. A total of 134 control serum samples were provided as blinded samples that were decoded upon completion of the analysis.

### Immunoassay Characteristics

Fifty micro liters of serum was diluted in 50 microliters of coating buffer (KPL, Gaithersburg, Md.) and plated in duplicates and in random order at room temperature overnight with shaking. Indirect ELISA assays for EPCA-2 were performed according to a standard protocol at 37° C. Inter-plate and intra assay variability for each assay was verified by plating various concentrations of the EPCA-2 peptides (0 to 1 mg per milliliters) and rabbit IgG in duplicates in each plate. To estimate the concentration of each sample, various concentrations (0.05, 0.1, 1, 10, 100 and 1000 ng per milliliters) of peptide controls for SPCA-2 were fitted on a sigmoidal dose-response curve using Graph Pad Prism 4 software, and the concentration was calculated from the equation generated by the curve. Once the EPCA-2 concentrations were calculated, they were compared with PSA levels.

### Statistical Analysis

The discriminatory accuracies of PSA and EPCA-2 were summarized using sensitivity, specificity, and Receiver Operating Characteristics (ROC) curves. All ROC curves and the areas under the curve were estimated empirically. Confidence intervals for sensitivities and specificities were exact binomial confidence intervals. Confidence intervals for estimated areas under the curves were asymptotic and based on the logistic transformation. See Pepe, M. S., THE STATISTICAL EVALUATION OF MEDICAL TESTS FOR CLASSIFICATION AND PREDICTION, Oxford University Press (2003). These formulations were used because many of the estimates were close to unity. When estimated areas under the curves were equal to 1, the inventors used the one-sided 95% confidence interval that has been shown to perform well in simulation studies. See Obuchowski et al., Acad. Radiol. 9: 526-30 (2002). This confidence interval is wider than or equal to that suggested by other methods for calculating confidence intervals in this situation. See Agresti et al., American Statistician 54: 280-88 (2000), and Reiser et al., Technometrics 28: 253-57 (1986).

For a threshold chosen based on pilot data, the inventors tested the joint null hypothesis that the sensitivity and specificity of EPCA-2 was no better than that of PSA (using a cut-off of 2.6 ng per milliliters). The null values of sensitivity and specificity were set to 0.81 and 0.41, respectively. See Thompson et al., JAMA 294: 66-70 (2005). The p-value for this hypothesis test was the smallest type-I error level for which the joint (rectangular) sensitivity/specificity confidence interval does not contain the null values. Finally, a paired t-test was used to test for a difference in mean EPCA-2 levels pre- and post-prostatectomy, using a random subset of the prostate cancer cases.

TABLE 1A
Serum sample profiles, mean PSA levels, mean EPCA-2.22, and mean Gleason scores.

| | Total No. | | | Mean | Mean | Mean |
|---|---|---|---|---|---|---|
| | of Samples | Age | | Total PSA | EPCA-2.22 | Gleason |
| Screening Population | (N = 178) | Range | Mean Age | (ng/ml) | (ng/ml) | Score |
| Normal (PSA < 2.5 ng/ml) | 33 | 51-82 | 64.59 ± 8.51 | 1.12 ± 0.59 | 15.18 ± 2.39 | N/A |
| Normal (PSA ≧ 2.5 ng/ml, | 30 | 45-71 | 60.70 ± 6.96 | 8.77 ± 6.66 | 14.12 ± 2.79 | N/A |
| negative biopsy for PCa) | | | | | | |
| BPH (Mean AUA symptom | 35 | 40-84 | 59.26 ± 10.58 | 1.34 ± 0.96 | 25.86 ± 4.72 | N/A |
| Score = 24) | | | | | | |
| PCa-OC | 40 | 45-67 | 57.38 ± 6.08 | 5.35 ± 2.53 | 33.90 ± 4.18 | 6.27 ± 0.45 |
| PCa-NOC | 40 | 43-70 | 58.60 ± 6.57 | 7.15 ± 4.69 | 42.81 ± 6.74 | 6.63 ± 0.74 |

PCa-OC: Organ confined prostate cancer;
PCa-NOC: Non-organ confined prostate cancer;
BPH: Benign Prostatic Hyperplasia.
Mean values are expressed with standard deviation (SD).

TABLE 1B
Serum sample profiles for the control groups
(other benign and cancer tissues).

| | No. of | | |
|---|---|---|---|
| | Samples | Patients Age | Mean |
| Control Population | (N = 134) | Range | EPCA-2.22 (ng/ml) |
| Normal female | 10 | 23-55 | 22.70 ± 3.152 |
| Benign breast | 9 | 36-68 | 22.00 ± 6.424 |
| Breast Cancer | 10 | 37-82 | 22.37 ± 5.62 |
| (Stage I-IV) | | | |
| Benign Colon | 9 | 42-75 | 19.58 ± 6.035 |
| Colorectal Cancer | 10 | 47-87 | 19.89 ± 5.259 |
| (Stage I-IV) | | | |
| Benign Liver | 10 | 27-74 | 20.82 ± 5.44 |
| Liver Cancer | 10 | 51-76 | 21.48 ± 5.688 |
| Benign Pancreatic | 10 | 17-85 | 19.58 ± 4.898 |
| Pancreatic Cancer | 10 | 60-80 | 22.14 ± 5.735 |
| (Stage I-IV) | | | |
| Benign Renal | 10 | 18-69 | 18.22 ± 6.046 |
| Renal Cancer | 9 | 45-71 | 16.99 ± 5.437 |
| (Stage I-IV) | | | |
| Bladder Cancer | 9 | 58-80 | 21.70 ± 4.675 |
| (Stage I-IV, | | | |
| PT1NoMo-PT3NoMx) | | | |
| Benign Lung | 9 | 35-71 | 18.48 ± 7.711 |
| Lung Cancer | 9 | 49-78 | 18.20 ± 5.979 |
| (Stage IB-IV) | | | |

Mean values are expressed with standard deviation (SD).

TABLE 1C
Clinicopathologic characteristics of healthy, BPH and PCa patients

| | Cohorts with PSA available | | | | |
|---|---|---|---|---|---|
| | Healthy | Healthy | | | |
| Characteristic | (PSA < 2.5 ng/ml) | (PSA > 2.5 ng/ml) | BPH | PCa (OC) | PCa (NOC) |
| Patients (n) | 43 | 30 | 33 | 43 | 45 |

Age

| | | | | | |
|---|---|---|---|---|---|
| Median (Range) | 59 (43-80) | 63 (45-71) | 58 (40-84) | 58 (45-67) | 60 (43-72) |
| Mean ± SD | 60.3 ± 8.6 | 60.7 ± 7.0 | 59.3 ± 10.9 | 58.3 ± 5.1 | 59.0 ± 6.4 |

Race (%)

| | | | | | |
|---|---|---|---|---|---|
| Caucasian | 38 (88) | 26 (87) | 18 (55) | 40 (93) | 43 (96) |
| African-American | 4 (9) | 3 (10) | 4 (12) | 2 (5) | 0 (0) |
| Other | 1 (2) | 1 (3) | 11 (33) | 1 (2) | 2 (4) |

PSA (ng/ml)

| | | | | | |
|---|---|---|---|---|---|
| Median (Range) | 1.0 (0.1-2.2) | 8.8 (2.5-39.9) | 1.2 (0.1-4.1) | 5.0 (1.6-11.8) | 5.9 (0.8-24.8) |
| Mean ± SD | 1.1 ± 0.6 | 8.8 ± 6.7 | 1.4 ± 0.9 | 5.2 ± 2.2 | 7.3 ± 4.6 |

EPCA-2.19 (ng/ml)

| | | | | | |
|---|---|---|---|---|---|
| Median (Range) | 0.11 (0.04-0.36) | 0.15 (0.07-0.43) | 0.23 (0.10-2.23) | 1.71 (0.27-22.72) | 2.11 (0.31-10.30) |
| Mean ± SD | 0.13 ± 0.07 | 0.18 ± 0.09 | 0.35 ± 0.37 | 2.94 ± 3.87 | 2.62 ± 2.27 |

Clinical Stage (%)

| | | | | | |
|---|---|---|---|---|---|
| cT1 | N/A | N/A | N/A | 36/41 (88) | 32/45 (71) |
| cT2 | N/A | N/A | N/A | 5/41 (12) | 13/45 (29) |
| cT3 | N/A | N/A | N/A | 0/41 (0) | 0/45 (0) |

Biopsy Gleason sum (%)

| | | | | | |
|---|---|---|---|---|---|
| 2-6 | N/A | N/A | N/A | 33 (77) | 27 (60) |
| 7 | N/A | N/A | N/A | 9 (21) | 16 (36) |
| 8-10 | N/A | N/A | N/A | 1 (2) | 2 (4) |

Prostatectomy Gleason sum (%)

| | | | | | |
|---|---|---|---|---|---|
| 2-6 | N/A | N/A | N/A | 32 (74) | 19 (42) |
| 7 | N/A | N/A | N/A | 10 (23) | 25 (56) |
| 8-10 | N/A | N/A | N/A | 1 (2) | 1 (2) |
| Extraprostatic extension | N/A | N/A | N/A | 0 (0) | 45 (100) |
| Seminal vesicle invasion | N/A | N/A | N/A | 0 (0) | 4 (9) |
| Lymph node invasion | N/A | N/A | N/A | 0 (0) | 1 (2) |
| Positive surgical margin | N/A | N/A | N/A | 2 (5) | 9 (21) |

TABLE 1D
Clinicopathologic characteristics healthy patients and patients with diseases other than
BPH and PCa

| | | Males | Mean age | Median age | Mean EPCA-2.19 | Median EPCA-2.19 |
|---|---|---|---|---|---|---|
| Patient Cohort | n | (%) | (yrs) ± SD | (yrs), Range | (ng/ml ± SD) | (Range) |
| Healthy Females | 10 | 0 (0) | 39.5 ± 11.0 | 43 (23-55) | 0.26 ± 0.12 | 0.23 (0.12-0.42) |
| Benign Breast | 9 | 0 (0) | 53.4 ± 11.3 | 50 (36-68) | 0.22 ± 0.14 | 0.18 (0.05-0.46) |
| Breast Cancer | 10 | 0 (0) | 55.4 ± 15.1 | 51 (37-82) | 0.15 ± 0.08 | 0.12 (0.06-0.34) |
| Benign Colon | 9 | 4 (44) | 59.2 ± 10.4 | 56 (42-75) | 0.21 ± 0.11 | 0.20 (0.06-0.39) |
| Colorectal Cancer | 10 | 8 (80) | 63.4 ± 13.0 | 63 (47-87) | 0.25 ± 0.14 | 0.23 (0.05-0.46) |
| Benign Liver | 10 | 3 (30) | 51.6 ± 13.9 | 50 (27-74) | 0.23 ± 0.10 | 0.20 (0.09-0.41) |
| Liver Cancer | 10 | 9 (90) | 64.6 ± 9.4 | 66 (51-76) | 0.23 ± 0.11 | 0.23 (0.07-0.41) |
| Benign Pancreas | 10 | 6 (60) | 62.7 ± 18.9 | 64 (17-85) | 0.21 ± 0.13 | 0.18 (0.06-0.47) |
| Pancreatic Cancer | 10 | 7 (70) | 71.6 ± 6.7 | 71 (60-80) | 0.22 ± 0.13 | 0.18 (0.05-0.42) |
| Benign Renal | 10 | 4 (40) | 44.1 ± 15.1 | 45 (18-69) | 0.12 ± 0.06 | 0.10 (0.07-0.24) |
| Renal Cell Cancer | 9 | 8 (89) | 58.3 ± 12.7 | 62 (37-71) | 0.18 ± 0.13 | 0.16 (0.05-0.44) |
| Bladder Cancer | 9 | 8 (89) | 67.7 ± 8.5 | 69 (58-80) | 0.20 ± 0.11 | 0.22 (0.06-0.37) |
| Benign Lung | 9 | 9 (100) | 53.4 ± 12.3 | 56 (35-71) | 0.21 ± 0.08 | 0.22 (0.11-0.34) |
| Lung Cancer | 9 | 9 (100) | 65.2 ± 10.8 | 69 (49-78) | 0.19 ± 0.11 | 0.16 (0.08-0.40) |

TABLE 1E
Serum sample profiles among 10 men who underwent
radical prostatectomy for prostate cancer.

| | | | | | Time PSA |
|---|---|---|---|---|---|
| | | | | | measured |
| | Total PSA (ng/ml) | | EPCA-2.22 (ng/ml) | | post- |
| | Pre- | Post- | Pre- | Post- | prosta- |
| | Prostatec- | Prostatec- | Prostatec- | Prostatec- | tectomy |
| Patient ID# | tomy | tomy | tomy | tomy | (months) |
| 3547027 | 5.1 | <0.1 | 32.74 | 26.91 | 24 |
| 3516391 | 10.7 | <0.1 | 32.43 | 25.68 | 8.5 |
| 3318106 | 3.7 | <0.1 | 32.95 | 23.25 | 14 |
| 1537471 | 6.9 | <0.1 | 30.51 | 26.84 | 24 |
| 3497772 | 7.6 | <0.1 | 32.17 | 28.20 | 17 |
| 2537112 | 5.7 | <0.1 | 32.46 | 24.00 | 8 |
| 3535480 | 5.4 | <0.1 | 32.72 | 25.36 | 19 |
| 3513900 | 3.4 | <0.1 | 30.74 | 21.59 | 8 |
| 3522674 | 6.1 | <0.1 | 32.12 | 19.36 | 23 |
| 3549860 | 13.1 | <0.1 | 31.08 | 30.53 | 14 |

TABLE 2A
Sensitivity and Specificity of PSA and EPCA 2.22. Proportions classified as
“positive”, according to PSA (using a cut-off of 2.5 ng/ml),
and EPCA 2.22 (using a cut-off of 30 ng/ml), along with 95% confidence intervals.

| | PSA | EPCA 2.22 |
|---|---|---|
| Specificity/Sensitivity | (2.5 ng/ml cut-off) | (30 ng/ml cut-off) |
| Specificity: Normal controls (PSA < 2.5 ng/ml) | 1.00 (95% CI 0.91 to 1.00)* | 1.00 (95% CI 0.91 to 1.00)* |
| Specificity: Normal controls (PSA < and ≧ 2.5 ng/ml) | 0.54 (95% CI 0.41 to 0.67) | 1.00 (95% CI 0.95 to 1.00)* |
| Specificity: Normal controls (PSA < and ≧ 2.5 ng/ml) and BPH controls | 0.65 (95% CI 0.55 to 0.75) | 0.92 (95% CI 0.85 to 0.96) |
| Specificity: Normal controls (PSA < and ≧ 2.5 ng/ml), BPH controls, and other controls | N/A** | 0.97 (95% CI 0.93 to 0.99) |
| Sensitivity: Organ-confined prostate cancer | 0.93 (95% CI 0.80 to 0.94) | 0.90 (95% CI 0.76 to 0.9 [symbol] |
| Sensitivity: Non-organ-confined prostate cancer | 0.90 (95% CI 0.76 to 0.97) | 0.98 (95% CI 0.87 to 1.00) |
| Sensitivity: Prostate Cancer-Overall | 0.91 (95% CI 0.83 to 0.96) | 0.94 (95% CI 0.86 to 0.98) |

*95% one-sided confidence interval
**PSA was not measured in the other control groups that include normal female as well as various benign and cancer serum samples [symbol]
indicates data missing or illegible when filed

TABLE 2B
Statistical analyses for EPCA-2.22.

| Test/Comparison | Significance |
|---|---|
| Hypothesis test for accuracy of EPCA-2.22 (30 ng/ml) | P = 0.002 |
| vs PSA (2.6 ng/ml¹³) | |
| Mean EPCA-2.22 levels before (31.99 ± 0.89 ng/ml) | P = 0.0002 |
| and after (25.17 ± 3.26 ng/ml) prostatectomy | |
| Mean EPCA-2.22 levels in PCa-OC (33.90 ± 4.18 ng/ml) | P < 0.001 |
| vs PCa-NOC (42.81 ± 6.74 ng/ml) | |

PCa-OC: Organ confined prostate cancer;
PCa-NOC: Non-organ confined prostate cancer.

TABLE 3
Specificity and Sensitivity of PSA and EPCA-2.19

| | Specificity ± SE (95% CI) | | |
|---|---|---|---|
| | PSA | PSA | EPCA-2.19 |
| Population (n) | (2.5 ng/ml cut-off) | (4.0 ng/ml cut-off) | (0.5 ng/ml cut-off) |
| Normal males and BPH (106) | 67 ± 4.6 (57-76) | 73 ± 4.3 (63-81) | 94 ± 2.2 (88-98) |
| Normal males, BPH and other diseases (240) | N/A | N/A | 98 ± 1.0 (95-99) |
| | Sensitivity ± SE (95% CI) | | |
| | PSA | PSA | EPCA |
| Population (n) | (2.5 ng/ml cut-off) | (4.0 ng/ml cut-off) | (0.5 ng/ml cut-off) |
| Prostate Cancer, OC and NOC (88) | 95 ± 2.2 (89-99) | 78 ± 4.4 (68-86) | 91 ± 3.1 (83-96) |
| Prostate Cancer, OC (43) | 93 ± 3.8 (81-99) | 67 ± 7.2 (51-81) | 88 ± 4.9 (75-96) |
| Prostate Cancer NOC (45) | 98 ± 2.2 (88-99) | 89 ± 4.7 (76-96) | 93 ± 3.7 (82-99) |

What is claimed is:

1. A method of analyzing a biological specimen for the presence of an analyte indicative of a clinical condition, comprising the steps of (A) contacting the specimen with an antibody that binds to the analyte and (B) detecting binding of the antibody in the specimen, wherein the antibody binds to an epitope comprised of an amino acid sequence selected from the group consisting of VIQPYPNFYMV and FAQDNDL, such that said binding is indicative of a cell-proliferative disorder of the prostate.

2. The method of claim 1, wherein the specimen is a prostatic tissue sample.

3. The method of claim 2, wherein the specimen is a biological fluid sample.

4. The method of claim 3, wherein the biological fluid sample is selected from among the group consisting of blood, plasma, serum, fecal matter, urine, semen, seminal fluid, and plasma.

5. The method of claim 4, wherein the biological specimen is serum.

6. Test kit for use in the method of claim 1, comprising (i) antibody that binds to an epitope comprised of an amino acid sequence selected from the group consisting of VIQPYPNFYMV and FAQDNDL, whereby said antibody forms a complex with EPCA-2 in a biological sample, and (ii) labeled anti-IgA or anti-IgG antibody for detecting said complex.

7. Test kit of claim 10, wherein said antibody is attached to a solid support.

8. Test kit of claim 6 or claim 7, further comprising reagents necessary for the detection of the label.

9. A method for determining the severity of a cell-proliferative disorder of the prostate, comprising: (a) contacting a biological specimen with an antibody that specifically binds to an epitope comprising the amino acid sequence VIQPYPNFYMV; and (b) determining the amount of protein comprising the epitope by detecting binding of the antibody in the specimen, wherein said amount of protein is indicative of the severity of the cell-proliferative disorder of the prostate.

10. The method of claim 9, wherein the specimen is a prostatic tissue sample.

11. The method of claim 9, wherein the specimen is serum.

12. A method of tracking the progression of prostate cancer over time, comprising: (a) determining the levels of EPCA-2 at a first time and a second time; and (b) comparing the levels at the EPCA-2 at the first and second times, wherein the level of EPCA-2 correlates with the progression of prostate cancer.
